(19) United States
(12) Patent Application Publication
(10) Pub. No.: US 2018/0344529 A1
(43) Pub. Date: Dec. 6, 2018

(54) EXPANDABLE EAR APPARATUS AND METHODS OF USE

(71) Applicant: MDIDEAFACTORY, INC., San Diego, CA (US)

(72) Inventors: Michael J. O'Leary, Del Mar, CA (US); Daniel Joseph Braun, San Diego, CA (US)

(21) Appl. No.: 15/993,396

(22) Filed: May 30, 2018

Related U.S. Application Data
(60) Provisional application No. 62/512,650, filed on May 30, 2017.

(51) Int. Cl. A61F 11/00; A61F 13/20; A61F 13/34; A61F 13/38

(52) U.S. Cl. CPC A61F 11/006; A61F 13/2017; A61F 13/38; A61F 13/2028; A61F 13/34

(57) ABSTRACT

Selectively expandable apparatus for insertion into an ear canal, and related methods of use and operation. In one embodiment, the apparatus includes a selectively expandable body configured for insertion into an ear and expansion within the ear canal. In one variant, the expanded main body is removable from the ear canal via a removal mechanism and removes excess cerumen and/or debris from the ear canal during removal. In another variant, the main body includes one or more compounds and is configured to receive a fluid for diffusion of the one or more compounds into the ear canal.

### PRIORITY AND RELATED APPLICATIONS

This application claims priority to U.S. Provisional Patent Application Ser. No. 62/512,650 filed May 30, 2017 and of the same title, which is incorporated herein by reference in its entirety. This application is related to PCT application Ser. No. ______ filed contemporaneously herewith on May 30, 2018 and of the same title, which is incorporated herein by reference in its entirety.

### COPYRIGHT

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

### 1. Technological Field

The disclosure relates to apparatus and methods for maintenance and use of the ear canal of a living being (such as a human), and in one exemplary aspect to an ear cleaning apparatus that is selectively insertable and removable for extraction of excess cerumen accumulation in the ear canal of a user. In another exemplary aspect, the disclosure relates to a compound delivery apparatus that is selectively insertable and removable for compound delivery (e.g., drug delivery) through an ear canal of the user.

### 2. Description of Related Technology

Many challenges exist in treatment of diseases of the ear (e.g. the human ear) and ear cleaning due to the ear having a delicate and complex arrangement of anatomical structures. Specifically, the auricle (i.e., the external portion of the ear) includes an opening to the auditory canal (meatus) where cerumen (i.e., ear wax) is secreted onto the skin surface. The auditory canal is limited at the internal end by the tympanic membrane (i.e., the ear drum), which prevents access to the middle and inner ear. Further, the inner ear is separated both physically and biochemically from systemic circulation via the blood labyrinth barrier (BLB). The inner ear is protectively isolated in the bony labyrinth with the exception of the round window membrane (RMW) which is a 2.5 mm² semi-permeable membrane. See FIG. 1.

The meatal orifice of the external ear is “guarded” from outside insect entry by a circumferential ring of fine hairs (to increase sensation) and cerumen-producing sebaceous glands located in the outer third of the cartilaginous ear canal. Cerumen is not produced deeper in the cartilaginous or bony ear canal. The sticky cerumen provides both a physical barrier to penetration of the canal as well as antimicrobial protection from both bacteria and fungi.

### Ear Cleaning and Maintenance

Ear cleaning apparatus and methods are also well known in the prior art. Such cleaning apparatus and methods include, for example, mechanical devices having a thin elongated shaft with a swabbing material disposed at one or both ends (e.g., a cotton swab stick), which is inserted into the ear and removes a portion of excess cerumen via manual wiping of the swabbing material against an inner surface of the auditory canal (see FIGS. 2A-2B). Other known methods include use of wire “loops”, wax solvents, hydraulic flushing, and ear candling.

Wire loops typically involve a small diameter wire or other element which can be inserted into the ear canal to remove cerumen; the distal end of the instrument (see FIG. 3) is blunted or rounded so as to help reduce the risk of perforation of the tympanic membrane.

Wax solvents are typically poured into an ear canal to chemically dissolve cerumen, while flushing includes spraying of fluid into the ear canal via a pressurized hydraulic stream (such as e.g., via a “water pick”).

Ear candling generally creates a thermal vacuum through a conical tube having one end inserted into an opening of the ear canal and an opposing end set aflame. See, for example, FIG. 4.

While widely used, the aforementioned apparatus and methods for ear cleaning may suffer from one or more of several deficiencies. Foremost, manual/mechanical swabbing and hydraulic flushing apparatus and methods often are inefficient in cerumen removal and push cerumen farther into the ear canal deepening the blockage and increasing the difficulty of removal. Moreover, such apparatus often use abrasive swabbing, mechanical scraping or spraying techniques, which may cause pain, ear drum perforation and, damage to the ear canal skin causing related disease (e.g., otitis externa) and hearing issues (e.g., cerumen occlusion of the ear canal and/or interference with the function of the tympanic membrane).

Other conventional methods, such as ear candling, can be equally ineffective and/or hazardous to the anatomy of the user (e.g., causing accidental burns to the auricle).

Further, although wax solvents may effectively dissolve cerumen, the liquefied cerumen then runs deeper into the ear canal towards the tympanic membrane, and can even cause irritation and/or infection.

### Delivery of Pharmaceutical Agents

Pharmaceutical agent (aka “drug”) delivery for treatment of conditions of the ear (external, middle and inner ear) is well known and is generally carried out systemically (e.g., oral, intravenous, and/or intramuscular drug delivery). However, numerous issues exist with systemic treatment of inner and/or middle ear disease and infection. Specifically, the BLB is penetrable to only a limited number of small lipid-soluble molecules. Further, systemic delivery typically requires high concentrations of drugs, such concentrations which may have deleterious side effects (e.g., organ damage, hypertension, arrhythmia, central nervous system excitation, hearing loss, etc.). Furthermore, using systemic delivery mechanisms, drug concentration is administered at a peak-working dosage, which subsequently decreases to a trough-low dosage due to e.g., dissolution via gastric juices, or other mechanisms of the host (dependent on the systemic delivery mode). Therefore, dosage during treatment is inconsistent, and may be ineffective due to such reduction of concentration subsequent to the peak-working dosage period. Finally, since the ear exists on the periphery of the body, delivery of adequate drug concentrations exposes the entire body to the increased concentrations.

Topical delivery of medications is another alternative which places the highest concentrations immediately in the desired location. Moreover, since distribution is limited by diffusion, medication levels are often negligible in the circulation or other sensitive organs, i.e., liver or kidney. The thin soft tissue of the external ear skin and middle ear mucosa are ideal tissues for topical drug delivery.

Hence, based on the foregoing, what is needed is safe and effective apparatus and methods which can be used for ear cleaning. Ideally, the aforementioned apparatus and methods would be selectively insertable and removable, and additionally limit abrasion to, and/or a degree of penetration into, the ear canal.

Moreover, an improved method and apparatus for delivery of localized or systemic agents or substances via the ear is needed.

### SUMMARY

The present disclosure addresses the foregoing needs by providing, inter alia, an improved apparatus and methods for ear cleaning and/or localized drug (or other substance) delivery or therapy provision.

In a first aspect of the disclosure, an apparatus that is selectively expandable within an ear canal and is configured for insertion into, and removal from, the ear canal is described. In one embodiment, the apparatus comprises a selectively expandable body having a first end and a second opposing end, the first end configured for insertion through an opening of the ear canal.

In one variant, the body comprises a “main” body and is configured to be expandable from an initial compacted state for insertion to a subsequent expanded state for removal. In one implementation, the main body comprises a compacted open cell material.

In another variant, the body comprises a spiral configuration. In one implementation, the spiral configuration comprises an “Archimedes screw”-type configuration. In another implementation, the spiral configuration comprises a double-helical configuration. In even another implementation the spiral configuration comprises a conical helical configuration.

In another variant, the body comprises a substantially cylindrical configuration. In one implementation, the cylindrical configuration comprises a rounded shape at the first end. In another implementation, the cylindrical configuration comprises at least one lateral projection at the second opposing end.

In other variant, the body comprises a depth-limiting mechanism. In one implementation, the depth-limiting mechanism comprises an annular flange disposed at the second opposing end. In another implementation, the depth-limiting mechanism comprises at least one lateral projection disposed at the second opposing end.

In another variant, the apparatus further comprises a removal mechanism. In one implementation, the removal mechanism comprises a flexible cord attached at the second end of the main body, the flexible cord configured to be grasped by a user and pulled in a direction away from an ear of the user for removal of the apparatus.

In another implementation, the removal mechanism comprises an at least semi-rigid tab attached to the second end of the main body, the at least semi-rigid tab configured to be grasped by a user and pulled in a direction away from an ear of the user for removal of the apparatus.

In a further variant, the body comprises a carrier material, the carrier material being configured to carry one or more compounds for diffusion into the ear canal. In one implementation the one or more compounds comprise an antibacterial compound. In another implementation the one or more compounds comprise an anti-fungal compound.

In a second aspect, apparatus for localized drug or other substance delivery to an ear are disclosed. In one embodiment, the apparatus comprises a body that is insertable and removable from an ear canal, the main body comprising a first end and a second opposing end, the first end configured for insertion through an opening of the ear canal, the body further comprising a carrier material configured to carry one or more compounds for diffusion into the ear canal. In one variant, the apparatus further comprises a removal mechanism attached to the second opposing end. In another variant, the apparatus further comprises a depth-limiting mechanism proximal to the second opposing end.

In a further variant, the carrier material comprises a selectively expandable material.

In even another variant, the carrier material comprises a compressed open celled expandable material.

In a third aspect, apparatus for removal of excess cerumen from an ear canal are disclosed. In one embodiment, the apparatus comprises a main body that is insertable and removable from an ear canal, the main body comprising a first end and a second opposing end, the first end configured for insertion through an opening of the ear canal, the main body further comprising a selectively expandable material.

In one variant, the apparatus further comprises a removal mechanism attached to the second opposing end. In another variant, the apparatus further comprises a depth-limiting mechanism proximal to the second opposing end. In even another variant, the main body comprises a spiral configuration. In yet another variant, the main body comprises a cylindrical configuration having a rounded shape at the first end.

In a fourth aspect, a method of drug delivery to an ear canal is disclosed. In one embodiment, the method includes selectively inserting a main body of a drug delivery apparatus into the ear canal. In one variant, the method further includes enabling diffusion of one or more compounds into the ear canal from a main body of the apparatus, the main body comprised of a carrier material. In another variant, the method further includes stopping the insertion via a depth-limiting mechanism. In yet another variant, the method further includes removal of the main body from the ear canal via a removal mechanism.

In a fifth aspect, a method for removal of excess cerumen and/or debris from an ear canal is disclosed. In one embodiment, the method includes selectively inserting a main body of a cerumen removal apparatus into the ear canal. In one variant, the method further includes expanding the main body after insertion into the ear canal, the main body comprised of an expandable material. In another variant, the method further includes stopping the insertion via a depth-limiting mechanism. In yet another variant, the method further includes removal of the main body from the ear canal via a removal mechanism.

Other features and advantages of the present disclosure will immediately be recognized by persons of ordinary skill in the art with reference to the attached drawings and detailed description of exemplary embodiments as given below.

### BRIEF DESCRIPTION OF THE DRAWINGS

FIG. 1 is a cross-sectional view of a typical human ear, illustrating various anatomical features thereof.

FIGS. 2A and 2B are cross-sectional views showing use of a typical swab-type of prior art ear canal cleaning device.

FIG. 3 is a perspective view of a “wire loop” type of prior art ear canal cleaning device.

FIG. 4 is a perspective view of an “ear candle” type of prior art ear canal cleaning device.

FIG. 5 is a perspective view of exemplary selectively expandable ear apparatus according to the present disclosure.

FIGS. 6A-6C are perspective views of various exemplary spiral (Archimedes screw) configurations for the ear apparatus of the present disclosure.

FIGS. 7A and 7B are perspective views of various exemplary spiral (double helical configurations for the ear apparatus of the present disclosure.

FIGS. 8A-8C are perspective and side elevation views, respectively, of another exemplary selectively expandable ear apparatus according to the present disclosure.

FIGS. 9A-9D are schematic views of an exemplary method of use of a selectively insertable ear apparatus according to the present disclosure.

FIG. 10 is a logical flow diagram of an exemplary method of use of a selectively insertable ear apparatus according to the present disclosure.

All figures © Copyright 2015-2016 M D Idea Factory. All rights reserved.

### DESCRIPTION OF THE DISCLOSURE

Reference is now made to the drawings wherein like numerals refer to like parts throughout.

### Overview

In one aspect, improved apparatus for ear canal cleaning, and related methods of use and operation are disclosed herein. In one embodiment, the apparatus includes a selectively expandable main body that is configured to be insertable and removable within an ear canal of a user.

In one example, the apparatus is inserted into a user's ear canal while a main body of the apparatus is in a compacted configuration (e.g., compacted in at least one dimension relative to its non-compacted state). After insertion, the apparatus is exposed to a fluid (e.g., water, saline solution, antiseptic, etc.) and maintained within the ear canal for a period of time within which the main body expands from the compacted position to an expanded position. When a desired expansion period has passed (i.e., a period within which the main body expands to contact surfaces of the ear canal and/or partially dries), the apparatus is removed from the ear canal via a removal mechanism. As the expanded apparatus is removed, outer surfaces of the main body slide over the surfaces of the ear canal, thereby removing excess cerumen and/or debris from the ear canal. The exemplary embodiment of the apparatus may be purposely designed to avoid deep insertion into the ear canal (where, as discussed above, deep insertion can cause potential damage and/or infection of the ear).

In another aspect, improved apparatus for compound delivery through an ear canal, and related methods of use and operation are disclosed herein. In one embodiment, the apparatus includes a selectively expandable main body that is configured to be insertable and removable within an ear canal of a user.

In one example, the apparatus is inserted into a user's ear canal while a main body of the apparatus is in a compacted configuration. In one implementation, the main body can be comprised of a carrier material having one or more compounds (e.g., drugs) disposed on a surface or within the carrier material. After insertion, the apparatus is exposed to a fluid (e.g., water, saline solution, etc.) and maintained within the ear canal for a period of time within which the main body expands from the compacted position to an expanded position. In another implementation one or more compounds are included in the fluid. In both implementations, addition or exposure of fluid enables diffusion of the one or more compounds from the main body of the apparatus to the skin of the ear canal. When a desired diffusion period has passed (i.e., a desired dosing period of the one or more compounds has passed), the apparatus is removed from the ear canal via a removal mechanism. As in the example above, the exemplary embodiment of the apparatus may be purposely designed to avoid deep insertion into the ear canal.

### Anatomy—

A brief discussion of the exemplary human ear anatomy is useful in further explanation of the various features and advantages of the apparatus and methods of the present disclosure.

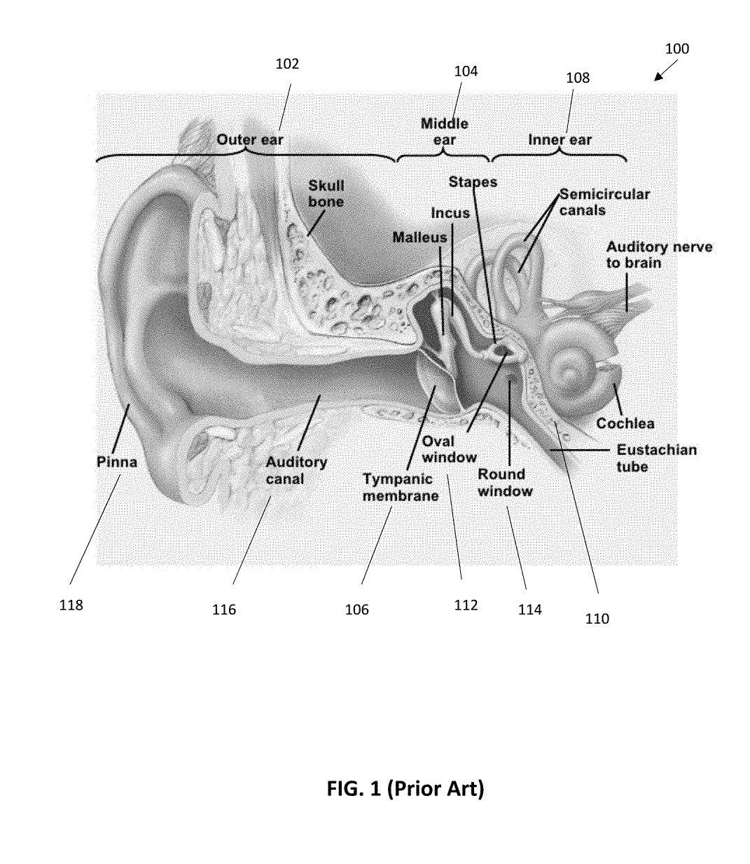

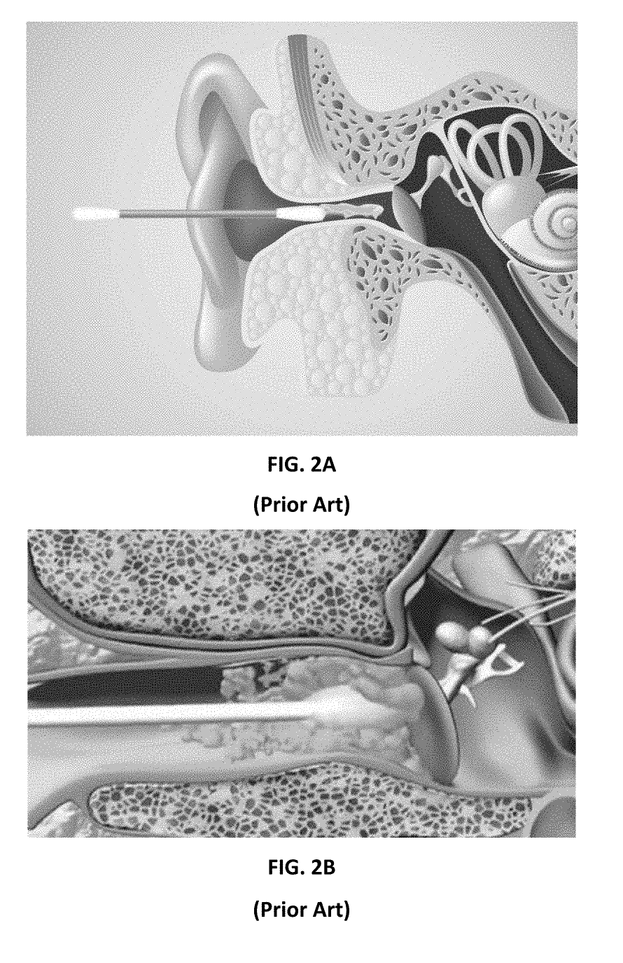

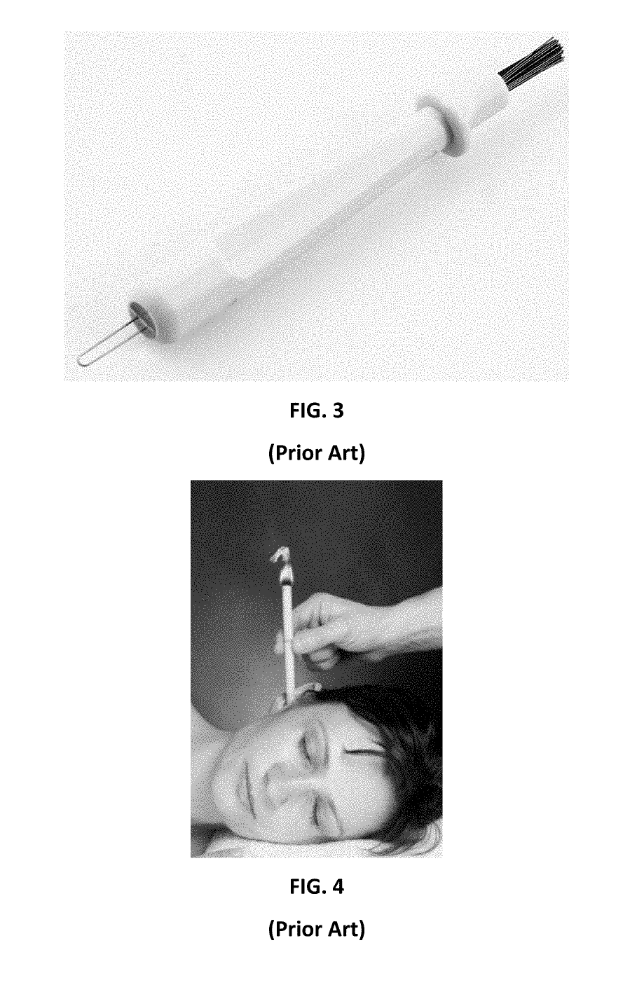

Referring now to FIG. 1, a cross section of a typical human ear 100 (viewed from a front perspective) is shown. In general, the ear includes an outer (external) ear 102, a middle ear 104 separated from the outer ear by a tympanic membrane (TM) 106, and an inner ear 108 separated from the middle ear by a wall of boney tissue 110 including two small 2.5 mm² “windows”—an oval window footplate at the base of the stapes 112 and a soft tissue round window membrane (RWM) 114.

Cerumen (i.e., ear wax) is a waxy substance secreted by sebaceous glands in the outer third of the cartilaginous external ear canal 116 of the external ear 102 It consists of triglycerides, wax esters, “squalene” (e.g., C₃₀H₅₀/(6E,10E,14E,18E)-2,6,10,15,19,23-Hexamethyltetracosa-2,6,10,14,18,22-hexaene), and free fatty acids mixed with desquamated skin cells. Excess or compacted cerumen can press against the tympanic membrane 106 or block the ear canal 116, thereby potentially causing pain and/or partial to complete hearing loss in the affected ear.

Additionally, as discussed elsewhere herein, the inner ear 108 includes a blood-labyrinth barrier (BLB) for regulation of homeostasis (e.g., volume and/or concentrations of fluid and ions) of the inner ear internal chambers or lumen (e.g., ducts of the cochlea and vestibular system). The RMW 114 acts as a semi-permeable membrane allowing some compounds (e.g., sodium ions, antibiotics, antiseptics, local anesthetics, etc.) to enter the perilymph and endolymph fluids of the inner ear 108 from the middle ear 104, while the TM 106 acts as a semi-permeable membrane between the outer ear 102 and the middle ear 104.

### Description of Exemplary Embodiments

It is noted that while the apparatus of the disclosure described herein are discussed primarily with respect to use in a medical context, such as for use in a medical setting (e.g., doctor's office, hospital, etc.), certain aspects of the disclosure may be useful in other applications, including, without limitation, consumer use (e.g., in a home setting).

It is also appreciated that while described herein primarily in the context of drug or other compound delivery intended to treat conditions of the middle or inner ear, the delivery methods and apparatus described herein are not so limited, and in fact may be used for delivery of other types of compounds, substances or agents (pharmaceutical or otherwise), and for treatment of conditions associated with other parts of the host organism. Such other compounds, substances and/or agents may include, without limitation and purely by way of example, steroids, growth factors (e.g., hepatocyte growth factor or HGF), anti-microbials, genetic vectors, nano-particles, hormones, and vitamins. Notably, the ear provides a pathway to the CNS (central nervous system) of the host, which can be utilized for delivery of therapy agents or other forms of treatment or stimulation targeting the CNS.

It is also noted that while the ear apparatus of the disclosure is described herein primarily in the context of a human wearer or user, many of the principles and features of the disclosure are adaptable to other species and their particular anatomical features by one of ordinary skill given the present disclosure including, without limitation, primates such as e.g., chimpanzees.

### Exemplary Apparatus—

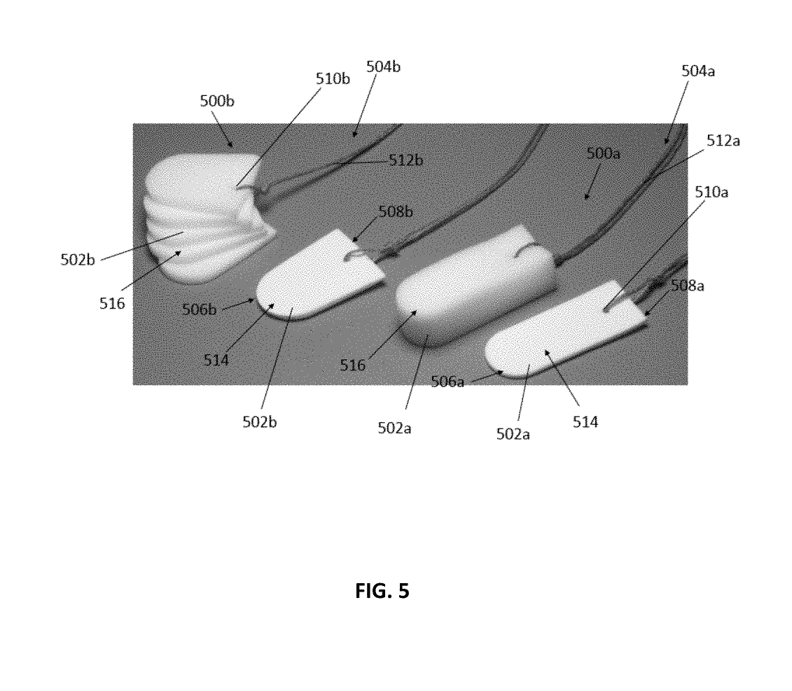

Referring now to FIG. 5, exemplary embodiments of the selectively expandable ear apparatus 500 (500a, 500b) of the present disclosure is shown and described.

As illustrated in FIG. 5, the exemplary apparatus 500a and 500b each include a main body 502 (502a, 502b) and a removal mechanism 504 (504a, 504b). In both embodiments, the main body 502 includes an elongated shape having a rounded first end 506 (506a, 506b) configured for insertion through the opening of the ear canal and a second opposing end (508a, 508b) configured for attachment of the removal mechanism 504 thereto. Specifically, in the examples shown in FIG. 5, the second end 508 includes a hole 510 (510a, 510b) configured for insertion of a flexible cord 512 (512a, 512b). The flexible cord (i.e., removal device) may be comprised of cotton thread and/or string, wire, plastic filament, and/or any other flexible or semi-flexible thin elongated material known or yet to be discovered.

Also illustrated in FIG. 5, the main body 502 is comprised of an expandable material and is thus selectively expandable from a compacted configuration 514 (i.e., dehydrated configuration) to an expanded configuration 516 (i.e., hydrated configuration). In one example, the main body is comprised of an expandable open cell material and/or hydromorphic polymer that expands when it comes into contact with fluid (e.g., water, saline solution, etc.). In one specific implementation the main body 502 is comprised of open-celled polyvinyl alcohol (PVA). Further, the open-celled PVA (or similar material) can be a carrier material for one or more compounds (e.g., drugs). The one or more compounds can be infused throughout the carrier material and/or disposed on a surface of the carrier material. Non-limiting example compounds that can be disposed within or on the carrier material include anti-bacterial compounds, anti-fungal compounds, steroids, hormones, or any other compound, including those for treatment of allergies, skin conditions (e.g., eczema), or even trauma/hearing loss to the affected ear(s).

In one implementation, compounds that are stable in a dehydrated state and active in a hydrated state are used; however, it will be appreciated that this is in no way a requirement, and in fact any number of different “original” and “delivery” states may be used consistent with the present disclosure, including e.g., those which are active in both a dehydrated and hydrated state, those activated by reaching a prescribed temperature (e.g., on the order of 98 degrees F. as is typically present in the tissues of the human ear canal), external heating (e.g., use of a blow dryer or other heated air source which gently and controllably raises the temperature of the ear tissue (and inserted apparatus), introduction of an external substance after the apparatus with medicant or agent is inserted into ear (e.g., introduction of an “activator” via an aerosolized spray, squeeze bottle, etc.), or by the presence of natural or other substances present in the user's ear (e.g., triglycerides, alcohols, squalene, etc.).

It will also be appreciated that the exemplary apparatus and methods described herein may be used for delivery of viscous or even non-liquid substances (whether in an activated or deactivated state), such as e.g., powders or other materials which can be assimilated and diffused by the ear tissue. For example, some organic materials may be directly diffused into the tissue without need for any “liquid” carrier per se. Moreover, some substances may be absorbed after volatilization thereof (e.g., change from liquid to vapor or aromatic state due to, e.g., introduction of a activator or even merely the presence of normal human body temperature within the ear canal).

It is also appreciated that the exemplary apparatus and methods described herein may be used for delivery of therapy agents or other substances targeted for the tympanic membrane and/or other portions of the ear canal, including the middle or even the inner ear, such as via diffusion or permeation through the membrane for e.g., the treatment of otitis media.

Advantageously, topical application of compounds, substances or agents to tissue such as that of the human ear often enable a great degree of variability in dosage; i.e., are very “forgiving” in terms of e.g., over-application or dosing, especially as compared to systemic/orally ingested approaches. Stated simply, it is harder to “overdose” a patient via topical introduction, especially when considering the limited surface area of the ear canal, and the volume of agent or compound that feasibly can be delivered at any given time via the exemplary apparatus described herein. This underscores another salient advantage of the various aspects of present disclosure; i.e., the need for less precision in dosing/application than with other approaches with respect to many types of compounds or agents being delivered.

Returning to FIG. 5, in the compacted configuration 514, each of the main bodies 502a and 502b are thin, substantially planar (i.e., two-dimensional) insertable tabs. As shown in FIG. 5, the main body 502a has a slightly greater length and a smaller width than the main body 502h In the expanded configuration, each of the main bodies 502a and 502b are generally expanded in the vertical direction and have an increased height as compared to the compacted configuration. As depicted in FIG. 5, the main body 502a is a single piece, while main body 502b includes multiple (i.e., 5) separate layers of expanded material.

It will be appreciated that while the body is shown as being generally flat and having a rounded end for insertion into the opening of the ear canal, other configurations may be used as well (and as described in greater detail subsequently herein), including without limitation an Archimedes screw spiraled configuration, other spiraled configurations (e.g., helical ribbon, twisted ribbon, etc.), a more cylindrical shape, a contoured shape (e.g., one or more regions that vary in diameter or other shape parameters, including e.g., linear or non-linear taper rates), and even asymmetric shapes. Further, the main body can have additional surface features, such as e.g., projections, raised portions, ridges, and/or otherwise textured surfaces, etc.

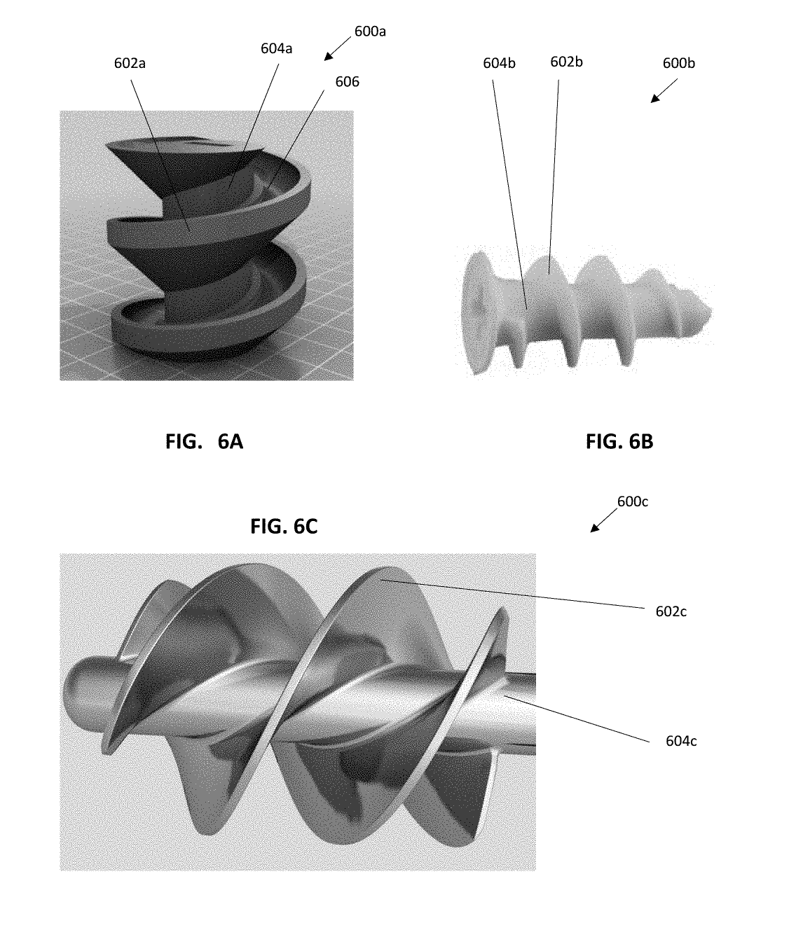

For example, FIGS. 6A-6C show three exemplary Archimedes screw-type spiral configurations 600 (600a, 600b, 600c) that may be used with the selectively expandable ear apparatus of the present disclosure. Each of the spiral configurations 600 includes a helical surface 602 (602a, 602b, 602c) disposed on and surrounding a central shaft 604 (604a, 604b, 604c). As depicted in FIG. 6A, in one example, the helical surface 602a has a substantially non-tapered configuration and includes a central trough. The example in FIG. 6B includes a tapered helical surface 602b, which is tapered (i.e., narrower) at the first end (i.e., insertion end) of the main body. Lastly, the example of FIG. 6C includes a slight taper at both the first end and the second end of the helical surface 602c. In one variant, the screw is formed of a somewhat rigid but pliable material (e.g., open-celled material previously described, or even a non-activated material which generally maintains its size and shape when inserted), and includes a substantially rounded insertion end so as to, inter alia, avoid damage to the tympanic membrane in the event of over-insertion.

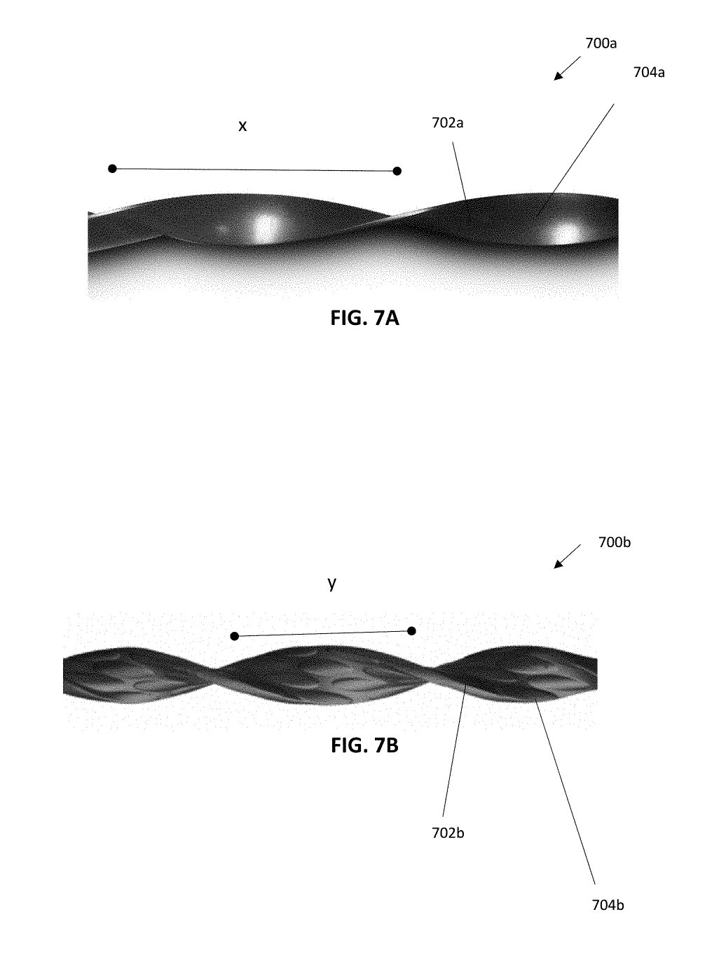

In another example, FIGS. 7A-7B show four exemplary other spiral configurations 700 (700a, 700b) that may be used with the selectively expandable ear apparatus of the present disclosure. Each of the spiral configurations 700 includes a ribbon-like body 702 (702a, 702b) twisted and regular intervals. As shown in FIGS. 7A and 7B, the ribbon-like body 702a has a “tighter” twisted configuration having a length v for a full turn of the body which is shorter than a length x of a full turn of the “looser” twisted configuration of the ribbon-like body 702b. Further, a surface 704a of the ribbon-like body 702a is substantially smooth, while the ribbon-like body 702b includes a textured surface 704b (having oval-shaped raised portions). It will also be appreciated that other symmetric or non-symmetric surface and/or shape features may be used, including e.g., ribs, or “teeth” (e.g., cone-shaped structures disposed normal to the local surface), rings, etc., whether alone or in combination with the foregoing spiral structure.

### Other Embodiments and Features

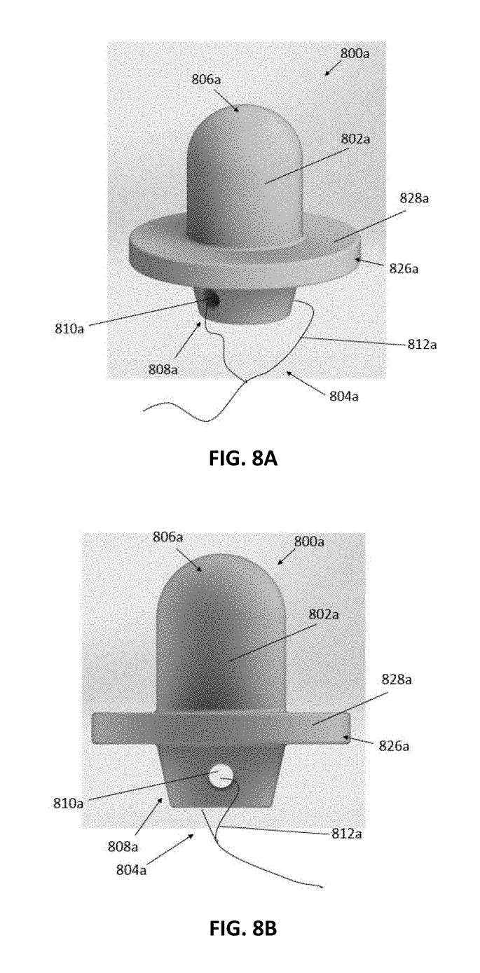

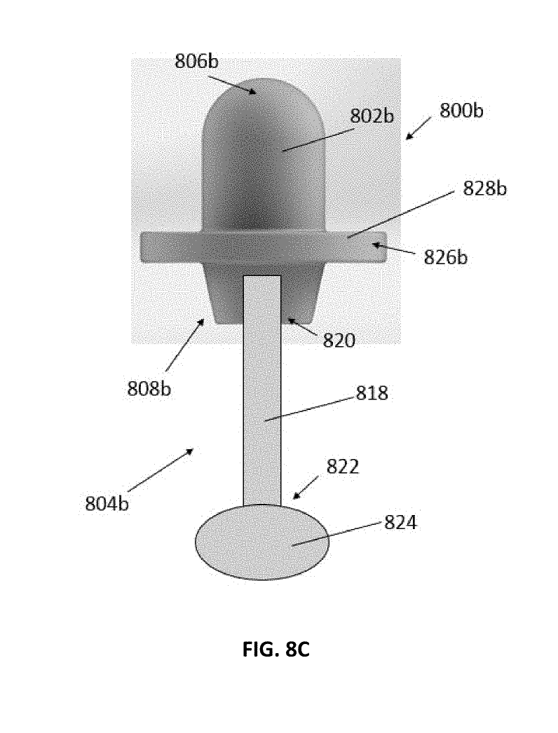

In even another example, FIGS. 8A-8C show exemplary selectively expandable ear apparatus 800 (800a, 800b) having a cylindrical configuration. Similar to apparatus 500, the apparatus 800 each have a rounded first end 806 (806a, 806b) configured for insertion into the opening of the ear canal and a second opposing end 808 (808a, 808b) configured for attachment of the removal mechanism 804 (804a, 804b) thereto. Specifically, in the example shown in FIGS. 8A and 8B, the second end 808a includes a hole 810a configured for insertion of a flexible cord 812a. The flexible cord (i.e., removal mechanism) may be comprised of cotton thread and/or string, wire, plastic filament, and/or any other flexible or semi-flexible thin elongated material known or yet to be discovered (e.g., plastic, metal, etc.). FIG. 8C shows an alternative configuration for a removal mechanism. As depicted in FIG. 8C, removal mechanism 804b includes an at least partially rigid elongated member 818 having a first end 820 attached to the second end 808b of the main body 802b and having a user-graspable tab 824 at a second end 822 of the elongated member 818.

Alternate configurations for the removal mechanism above include e.g., elongated tabs at or extending from the outward/lateral (non-inserted) side of the apparatus, soft “hook” rings of a prescribed radius that can be grasped by the user or latched onto with an external implement, or even suction/vacuum on an external substantially smooth surface.

Similar to the apparatus 500, in the apparatus 800 the main body 802 is comprised of an expandable material and is thus selectively expandable from a compacted configuration (i.e., dehydrated configuration) to an expanded configuration (i.e., hydrated configuration). In one example, the main body is comprised of an expandable open cell material and/or hydromorphic polymer that expands when it comes into contact with fluid (e.g., water, saline solution, antiseptic solution, alcohol, etc.). In one specific implementation the main body 802 is comprised of open-celled polyvinyl alcohol (PVA). Further, the open-celled PVA (or similar material) can be a carrier material for one or more compounds (e.g., drugs). The one or more compounds can be infused throughout the carrier material and/or disposed on a surface of the carrier material. Non-limiting example compounds that can be disposed within or on the carrier material include anti-bacterial compounds, anti-fungal compounds, steroids, or any other compound that is stable in a dehydrated state and active in a hydrated state.

Moreover, exemplary uses of the apparatus described herein include treatment of eczema, hematoma, granulation, or even foreign bodies within the ear. Yet other uses contemplated by the inventors hereof include delivery/placement of markers for stereotactic radiosurgery, temperature probes, or even hearing or protection from mechanical and undesirable chemical agents, such as via absorption of external liquid substances, removal of toxins, pus, etc.

Returning to FIGS. 8A-8C, each of the main bodies 802 include a depth-limiting mechanism 826 (826a, 826b) configured to limit a depth of insertion of the main body into a user's ear canal. In the embodiments shown in FIGS. 8A-8C, the depth-limiting mechanism 826 includes an annular flange 828 (828a, 828b) which projects outwardly from the main body 802 in a lateral direction. The annular flange 828 is disposed between first end 806 and second end 808 and is proximal to second end 808. The depth-limiting mechanism may be comprised of the same material as the main body (e.g., open-cell PVA) or it may be comprised of a different material (e.g., silicone, plastic, etc.).

In alternate examples, the depth-limiting mechanism can have a different configuration such as, e.g., one or more projections extended outwardly from the main body in a lateral direction. Further, in additional alternate examples, the depth-limiting mechanism can be closer or farther from the first end, depending on a desired insertion depth. Furthermore, such depth-limiting mechanisms can be included with an apparatus having a planar configuration (such as e.g., planar configurations shown in FIG. 5) or a spiral configuration (such as, e.g., spiral configurations shown in FIGS. 6A-7B.

It will also be appreciated that the present disclosure likewise contemplates substantially customized apparatus which are formed so as to be anatomically optimized for each user or class of users (e.g., child-sized apparatus, adult-sized apparatus, etc.). Using e.g., well known three-dimensional (3D) optical or laser scanning apparatus (such as of the type used in conjunction with so-called “3D printers”), a 3D model of a given user's outer ear and outer canal can be obtained, and the molding or formation of the apparatus adjusted accordingly. In fact, since human beings are often somewhat asymmetric in terms of anatomical features (including ears), it is contemplated that a “left/right side” model can be used to customize apparatus for each ear (and e.g., labeled or color coded so as to permit ready insertion into the appropriate ear), as well as those having chirality (i.e., “handed-ness”).

Moreover, the present disclosure contemplates purchase and manufacture of such plugs in bulk for each user or class of users, such as for disposable apparatus, thereby assuring that the user will have a ready supply of customized apparatus, while taking advantage of manufacturing economies. In one such paradigm, a home or workplace user can simply utilize a hand-held optical or other scanner to scan an “earprint” of themselves, and send it to a remote entity via e.g., the Internet so as to effectuate the order and production of the customized apparatus.

### Description of Exemplary Methods—

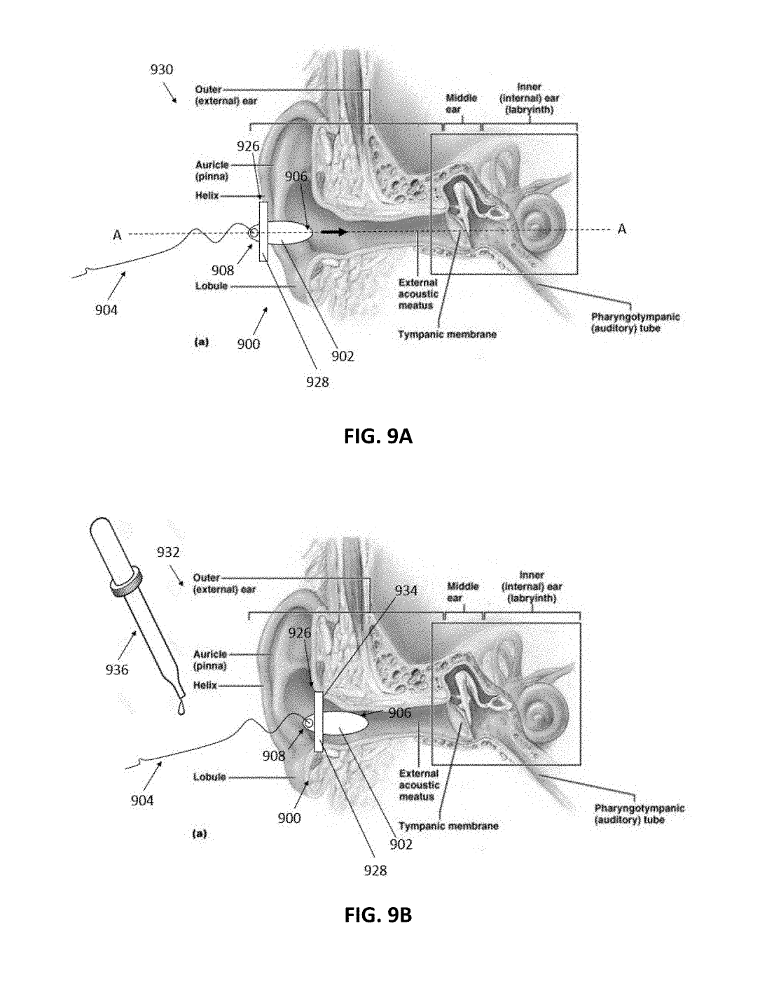

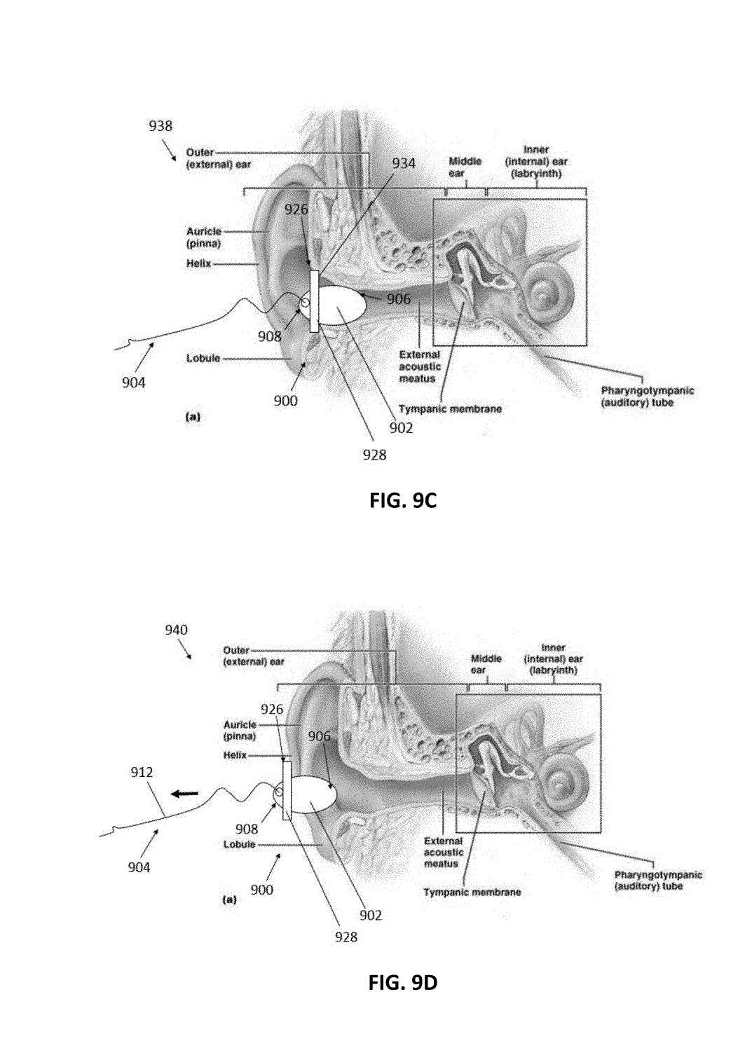

Turning now to FIGS. 9A-9D a schematic depiction of an exemplary method of use for one example embodiment selectively expandable ear apparatus 900. It will be appreciated that the depicted exemplary method may be used with any of the example ear apparatus described herein.

In FIG. 9A, apparatus 900 is shown in a pre-insertion and compacted/dehydrated state 930. For alignment and insertion of apparatus 900, a longitudinal axis A-A of a main body 902 is aligned with an ear canal of the user. Further, a first end 906 of the apparatus 900 is oriented toward the opening of the ear canal. The user or another individual (e.g., a doctor, a nurse, a parent, etc.) can grasp a second opposing end 908 and/or a depth-limiting mechanism 926 for manipulation of the apparatus 900 during alignment and insertion. Specifically, the user pushes the main body into the ear canal.

FIG. 9B shows apparatus 900 in an inserted and compacted/dehydrated state 932. In state 932, the main body 902 is disposed within the user's ear canal proximal to the ear canal opening. As shown in FIG. 9B, the main body 902 is disposed in the region of cerumen production and remains distal from the sensitive tympanic membrane. Further, an outer surface of the main body 902 does not contact or only partially contacts the skin surface of the ear canal. The depth of insertion into the ear canal is limited by the depth-limiting mechanism 926. Specifically, an inner surface 934 of an annular flange 928 is abutted to the auricle in a region proximal to the ear canal opening and limits further movement of the main body 902 deeper into the ear canal.

Also shown in FIG. 9B, after insertion of the main body into the ear canal, liquid (e.g., water, saline solution, antiseptic solution, alcohol, a solution containing one or more drugs, etc.) is dispensed onto the main body 902 via a fluid dispensing device 936. In the present example, the fluid dispensing device 936 is a dropper having an aspirating bulb. In other examples, the fluid dispensing device can be any device configured for controlled dispensing of liquid (e.g., dispensing relatively small amounts of liquid at a specific location). The fluid is dispensed from the fluid dispensing device onto the second end 908 and is drawn toward the first end 906 via capillary action (i.e., wicking).

FIG. 9C shows apparatus 900 in an inserted and expanded/hydrated state 938. In state 938, the main body 902 remains disposed within the user's ear canal proximal to the ear canal opening. As shown in FIG. 9C, the main body 902 is disposed in the region of cerumen production and remains distal from the sensitive tympanic membrane. Further, an outer surface of the expanded main body 902 contacts the skin surface of the ear canal. In one example, the main body 902 expands to the circumference of the ear canal and substantially fills the ear canal in the region proximal to the ear canal opening. The depth of insertion into the ear canal remains limited by the depth-limiting mechanism 926.

In examples where apparatus 900 is used for administering one or more compounds into the ear canal (e.g., drug delivery), the main body 902 can remain in the inserted and expanded/hydrated state 938 for a desired diffusion period. Further, additional fluid can be added to the main body during the diffusion period.

In exemplary implementations, agents or compounds that cannot tolerate the gastrointestinal tract (e.g., which would be adversely affected by stomach acids or other such substances prior to absorption via the small intestine), and hence otherwise require intravenous infusion or other approaches, are advantageously delivered via the apparatus and methods herein. For example, testosterone therapy, anti-cancer drugs, and antibiotics often meet such criteria. It is also noted that advantageously, the methods and apparatus described herein may be used to effect controlled release of the compound/agent that could last over several hours to several days or even weeks, to provide a sustained effect or achieve a desired profile of the substance within the user's tissue and/or blood stream (or CNS).

Otherwise, in examples where apparatus 900 is used for removal of excess cerumen from the ear canal, the main body 902 can remain in the inserted and expanded/hydrated state 938 for a desired expansion period. Specifically, it may be desirable to maintain the position of the main body until the main body is fully expanded and has slightly dried in the expanded configuration.

Lastly, FIG. 9D shows the apparatus 900 in a post-insertion (i.e., removed) and expanded/hydrated state 940. In state 940, the main body 902 is removed from the user's ear canal via the removal mechanism 904. Specifically, a user or another individual (e.g., doctor, nurse, parent, etc.) grasps a cord 912 of the removal mechanism and gently pulls on the cord in a direction away from the user's ear until the main body 902 is free of the ear canal. In examples where the apparatus 900 is used for ear cleaning, excess cerumen and/or other debris is transferred or caught on the outer surface of the main body 902 as the device is removed. In other words, the outer surfaces of the main body wipe or swab against the skin of the ear canal as the apparatus is removed, thereby removing cerumen and/or debris within a region of the ear canal proximal to the ear canal opening.

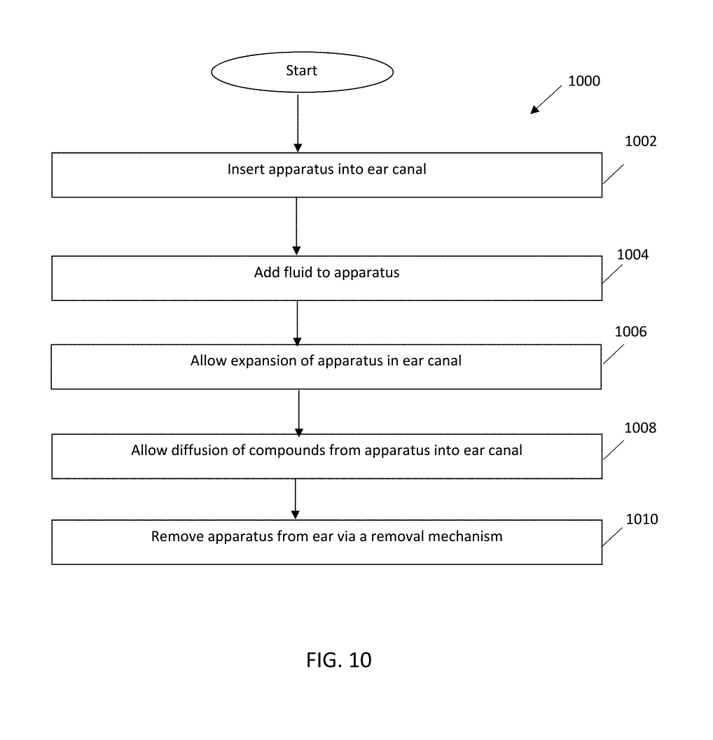

FIG. 10 shows a logical flow diagram of another exemplary method 1000 for use of the aforementioned selectively expandable ear apparatus. At step 1002, a main body of the apparatus is inserted into the ear canal of a user by the user or another individual (e.g., doctor, nurse, parent, etc.) via grasping of a second end and/or depth-limiting mechanism. As described with reference to FIG. 9A, the apparatus is in a compacted/dehydrated state and a longitudinal axis of the main body is aligned with the user's ear canal with a first end oriented toward the ear canal opening. In examples including a depth-limiting mechanism the apparatus is inserted until the depth limiting mechanism is abutted to the user's auricle.

At step 1004, the main body is maintained in the ear canal as fluid is added to the second end. The addition of fluid causes the main body to expand from the compacted/dehydrated state to an expanded/hydrated state. In the expanded/hydrated state, the main body expands to the circumference of the ear canal and substantially fills the ear canal in the region proximal to the ear canal opening at step 1006. In some examples, the position of the main body is maintained for a desired expansion period to allow the main body to expand and/or slightly dry.

Optionally, at step 1008, if one or more compounds are disposed within or on the main body and/or if one or more compounds are in solution in the fluid added the main body, the position of the main body in the ear canal can be maintained for a desired diffusion period. During the diffusion period, the one or more compounds diffuse from the main body into the skin surface of the ear canal. Additionally, the one or more compounds can diffuse from the main body to the ear canal and further to the tympanic membrane and/or to the round window membrane (RMW).

Finally, at step 1010 the main body is removed from the ear canal via a removal mechanism. Specifically, a user or the other individual grasps a portion of the removal mechanism a tab, a cord, etc.) and pulls on the removal mechanism in a direction away from the user's ear until the main body is free of the ear canal. In examples where the apparatus is used for ear cleaning, the outer surface of the main body wipes and/or swabs the skin of the ear canal as the main body is removed, thereby cleaning cerumen and/or debris from the ear canal.

It should be recognized that while the foregoing discussion of the various aspects of the disclosure has described specific sequences of steps necessary to perform the methods of the present disclosure, other sequences of steps may be used depending on the particular application. Specifically, additional steps may be added, and other steps deleted as being optional.

Furthermore, the order of performance of certain steps may be permuted, and/or performed in parallel with other steps. Hence, the specific methods disclosed herein are merely exemplary of the broader methods of the disclosure.

While the above detailed description has shown, described, and pointed out novel features of the disclosure as applied to various embodiments, it will be understood that various omissions, substitutions, and changes in the form and details of the device or process illustrated may be made by those skilled in the art without departing from the disclosure. The described embodiments are to be considered in all respects only illustrative and not restrictive. The scope of the disclosure is, therefore, indicated by the appended claims rather than the foregoing description. All changes that come within the meaning and range of equivalence of the claims are embraced within their scope.

What is claimed is:

1. Apparatus comprising a first element configured to at least partly fit within at least a portion of an ear of a living subject, the element configured to selectively expand in at least one dimension when exposed to an activator substance while in the ear, the selective expansion causing cerumen within the ear to adhere to the first element so as to enable subsequent removal of at least a portion of the adhered cerumen.

2. The apparatus of claim 1, wherein the activator substance comprises at least water, and the first element comprises a compressed material configured to expand in the at least one dimension when wetted by the water.

3. The apparatus of claim 3, wherein the activator substance comprises at least a solvent and a medicant, and the first element comprises a compressed material configured to expand in the at least one dimension when wetted by the solvent.

4. The apparatus of claim 1, wherein the first element comprises at least:
a main body configured for insertion into the at least portion of the ear; and
a removal mechanism attached to at least a portion of the main body, the removal mechanism configured to extend at least partly outside of the at least portion of the ear in a condition where the main body is inserted into the portion of the ear.

5. The apparatus of claim 4, wherein the main body comprises a rounded distal end, the rounded distal end disposed at an opposing end of the main body relative to the removal mechanism.

6. The apparatus of claim 4, wherein the main body comprises a spiral configuration.

7. The apparatus of claim 4, wherein the main body comprises a cylindrical configuration.

8. The apparatus of claim 4, wherein the main body comprises a textured surface.

9. The apparatus of claim 4, wherein the removal mechanism comprises a user graspable portion configured to enable a user to pull the main body out of the at least portion of the ear after expansion of the compressed material.

10. The apparatus of claim 9, wherein the user graspable portion comprises an elongated flexible filament.

11. The apparatus of claim 9, wherein the user graspable portion comprises an elongated tab.

12. A method for treating at least one condition of an ear, the method comprising:
inserting a main body of an ear treatment apparatus into at least a portion of a canal of the ear, the main body being in a compacted configuration during the inserting;
enabling a removal mechanism to remain outside of the at least portion of the canal;
exposing at least the main body to an activator substance, the activator substance causing the main body to expand in at least one dimension thereby changing to an expanded configuration while disposed in the at least portion of the ear canal; and
utilizing the removal mechanism to pull the main body from the at least portion of the ear canal, the main body being in the expanded configuration while pulled from the at least portion of the ear canal.
